(19) United States
(12) Patent Application Publication
(10) Pub. No.: US 2003/0095954 A1
(43) Pub. Date: May 22, 2003

(54) Novel method for promotion of angiogenesis

(76) Inventors: Hiroyuki Koyama, Tokyo (JP); Hiroshi Shigematsu, Tokyo (JP); Tetsuro Miyata, Tokyo (JP)

Correspondence Address: Robert G. Mukai, BURNS, DOANE, SWECKER & MATHIS, L.L.P., P.O. Box 1404, Alexandria, VA, 22313-1404, US

(21) Appl. No.: 10/156,135

(22) Filed: May 29, 2002

(30) Foreign Application Priority Data
Nov. 21, 2001 (JP) 2001-356,765
May 29, 2002 (JP) 2002-154,960

(51) Int. Cl. A61K 48/00; C12N 15/861

(52) U.S. Cl. 424/93.21; 435/456; 435/320.1

(57) ABSTRACT

A novel method for promotion of angiogenesis and arteriogenesis is provided. This invention provides a novel method for promotion of angiogenesis and arteriogenesis, wherein a growth factor gene is introduced into fibroblasts ex-vivo using adenovirus vector. Moreover, the method according to this invention can improve cardiac blood flow rate of ischemic region, thereby a novel method for treatment of ischemic heart disease is also provided.

### BACKGROUND OF THE INVENTION

1. Field of the Invention

This invention relates to a novel method for promotion of angiogenesis and arteriogenesis, wherein growth factor gene is introduced into fibroblasts ex-vivo using adenovirus vector. Moreover, this invention relates to a novel method for treatment of ischemic heart disease by improving cardiac blood flow rate of ischemic region.

2. Description of the Prior Art

Accompanied with increased arterial sclerosis caused by progressive aging of society, the number of patients in need with revascularization therapy is increasing. Operation techniques and materials have achieved progression and the result of surgical revascularization has been improved. However, as to diseases such as peripheral occluded artery complicated with diabetes mellitus, cases inapplicable of severely invasive surgery for complicated diseases and peripheral Buerger's disease, revascularization therapy remains to be inapplicable yet. For such patients, medicines such as vasodilator, platelet aggregate inhibitor or the like have been administrated. However, such therapy has certain limit on its efficacy and the patients are forced to amputate their legs. Meanwhile, when arterial occlusion occurs, living bodies can auto-develop collateral artery to recover blood circulation to some extent. If the mechanism involved in development of collateral artery is elucidated, development of collateral artery can be achieved by further promotion of angiogenesis and arteriogenesis, thereby ischemia in inferior limb would be improved. Then, it would provide an effective therapeutic method for cases surgical revascularization could not be applied and clinical therapy with conception of “therapeutic collateral development” would be realized.

It has been known that angiogenesis and arteriogenesis can be induced using growth factors such as “acidic fibroblast growth factor (aFGF)”, “basic fibroblast growth factor (bFGF)”, vascular endothelial growth factor and hepatocyte growth factor. In basic animal experiments using inferior limb ischemia models and cardiac ischemia models, these growth factors have been administrated through various routes in the early 1990s and superior collateral development has been recognized. In the early stage, growth factor proteins have been directly administrated into rabbit arteries to evaluate the effect on collateral development in rabbit model animals of hind limb ischemia. Then significant development of collateral arteries and improvement in ischemia has been reported. However, in the case sufficient amount of growth factor protein for angiogenesis and arteriogenesis were administrated into arteries of the animal all at once, high concentration of growth factor protein would distribute in the body through systemic blood flow. Then occurrence of undesirable side effects caused by the administrated high concentration proteins would be worried.

As to another method for induction of therapeutic collateral development, gene incorporation of a growth factor gene can be mentioned. Cells transfected by the growth factor gene can continuously secret growth factor protein for a certain period. Thus when the growth factor gene was introduced into arterial wall cells or muscle cells of ischemic tissue, significant development in collateral arteries has been recognized. However, direct introduction into arterial wall cells is would have difficulties for the purpose of practical clinical application. As arterial lesions with severe ischemia are extended all around and complicated in general, the introduced gene has difficulties in reaching to the target site. In addition, affected arteries have already shown arterial sclerosis and efficacy in gene incorporation would be decreased. Meanwhile, Tsurumi et al succeeded in transfection of the VEGF gene to muscle cells by direct intramuscular injection of naked DNA. This method utilized the unique profile of muscle cells that take up and express a foreign gene transferred in the form of naked plasmid DNA. Since the VEGF gene was directly injected intramuscularly, thus the administration method is simple and easy. Therefore, there would be less limitation on its clinical application to human inferior limb ischemia.

### BRIEF DESCRIPTION OF THE INVENTION

Therefore, there is a strong demand on development of a method for gene therapy with higher efficacy and safety, directed to angiogenesis and arteriogenesis against chronic inferior limb ischemia. This invention provides a novel method for gene introduction, based on the knowledge obtained from investigation on development of collateral arteries in model rabbits of inferior limb ischemia.

Therefore, this invention relates to a method for promotion of angiogenesis and arteriogenesis, the method comprises the steps of;

(1) preparing modified adenovirus vector by incorporating growth factor gene fused with secretory signal sequence into an adenovirus vector,

(2) obtaining non-hematocytes from a creature being target of angiogenesis and arteriogenesis, and culturing said non-hematocytes ex-vivo,

(3) infecting cultured said non-hematocytes with said modified adenovirus vector to prepare non-hematocytes having growth factor secretory ability, by introducing said modified adnovirus vector into said non-hematocytes; and

(4) administrating said non-hematocytes having growth factor secretory ability via blood vessel of said creature, thereby said growth factor is secreted in body of said creature.

These and other advantages of this invention will become apparent upon a reading of the detailed descriptions and drawings.

### BRIEF EXPLANATION OF DRAWINGS

FIG. 1 is a photograph showing the result of Western blot analysis of culture medium of virus infected fibroblasts.

FIG. 2 is a photograph showing the result of Western blot analysis of cell lysate of virus infected fibroblasts.

FIG. 3 is a graph showing time course of bFGF expressed in culture medium of rabbit fibroblasts.

FIG. 4 is a graph showing time course of bFGF expressed in cell lysate of rabbit fibroblasts.

FIG. 5 is a graph showing mitotic activity of bFGF secreted into culture medium measured by ³H-thymidine method.

FIG. 6 is a schematic figure showing experimental protocol designed for hind limb ischemic model.

FIG. 7 is a schematic figure showing experimental protocol designed for hind limb non-ischemic model.

FIG. 8 is a graph showing distribution of ¹¹¹In-labeled fibroblast in organs and tissues at 5 hour after cell injection into the left internal iliac artery.

FIG. 9 is a graph showing correlation between cell distribution (%) of left ventricle injected ¹¹¹In-labeled fibroblast into bilateral hind limb muscles and regional blood flow measured with ⁵¹Cr-labeled microspheres.

FIG. 10 is a graph showing cell distribution after administration into the left ventricle and regional blood flow in bilateral hind limb muscles.

FIG. 11 is a graph showing calf pressure ratio immediately after femoral artery excision, immediately before, immediately after and 28 days after cell administration.

FIG. 12 is a photograph showing selective internal iliac angiograms of AxCALacZ virus injected rabbit at 28 days after injection of infected fibroblasts.

FIG. 13 is a photograph showing selective internal iliac angiograms of AxCAMAssbFGF virus injected rabbit at 28 days after injection of infected fibroblasts.

FIG. 14 is a graph showing development of collateral vessels quantified by the angiographic score 28 days after cell injection.

FIG. 15 is a graph showing capillary density.

FIG. 16 is a graph showing diameter of proximal left gluteal artery.

FIG. 17 is a graph showing blood flow of left internal iliac artery at rest and maximum.

FIG. 18 is a graph showing time course of bFGF blood concentration measured by ELISA method.

FIG. 19 is a graph showing anti-adenovirus antibody titer in blood.

FIG. 20 is a photograph showing bFGF-positive left adductor muscle 4, 7, 14 and 28 days after injection of AxCAMAssbFGF virus injected cells.

FIG. 21 is a photograph showing time-course of bFGF accumulation in left adductor muscle, lung and liver.

FIG. 22 is a graph showing alteration of calf blood pressure ratio immediately after femoral artery excision, and immediately before, immediately after and 28 days after cell administration at groups of respective cell numbers.

FIG. 23 is a photograph showing the result of selective internal iliac angiograms at vehicle group and groups of respective cell numbers.

FIG. 24 is a graph showing (a) in vivo blood flow of left iliac artery at rest and (b) maximum in vivo blood flow of left internal iliac artery.

FIG. 25 is a graph showing capillary density of left semimembranous muscles measured in tissue sections stained by indoxy-tetrazolium method at groups of respective cell numbers.

FIG. 26 is a graph showing distribution of ¹¹¹In-labeled fibroblasts at 5 hours after injection of 1×10⁶ cells (a), 5×10⁶ cells (b) and 2.5×10⁷ cells (c) into the left internal iliac artery.

FIG. 27 is a photograph showing time course of bFGF accumulation in left adductor muscle (a), lung (b) and liver (c), analyzed by Western blotting using detection by anti-bFGF antibody.

FIG. 28 is a graph showing (a) time course of systemic bFGF level measured by ELISA and (b) time course of anti-adenovirus antibody titer quantified by neutralizing test.

FIG. 29 is a graph showing time course of (a) PaO₂ and (b) PaCO₂ in blood gas analysis and a photograph of lung with Elastica van Gieson staining in (c) AxCAMAssbFGF-treated and (d) AxCALacZ-treated rabbits.

FIG. 30 is a schematic figure showing the experimental protocol.

FIG. 31 is a bull's eye-like diagram representing the division of the left ventricle (LV).

FIG. 32 is a figure showing division of the LV and definition of the ischemic area in regional myocardial blood flow measurement.

FIG. 33 is (a) a photograph showing the result of Western blot analysis and (b) a graph showing the result of ³H-incorporation assay.

FIG. 34 is a graph showing the left ventricular ejection fraction (EF).

FIG. 35 is a figure showing three-dimensional local shortening (LS) maps of (a) before and (b) 28 days after fibroblast injection.

FIG. 36 is a graph showing the Rentrop scores of the bilateral coronary arteriography (CAG).

FIG. 37 is a photograph showing the right CAG in a pig belonging to the bFGF group obtained (PRE) before and (POST) 28 days after fibroblast injection.

FIG. 38 is a graph showing myocardial blood flow rate in the ischemic and non-ischemic areas 28 days after fibroblast injection.

### DETAILED DESCRIPTION OF THE INVENTION

The first feature of this invention is that growth factor gene for promotion of angiogenesis and arteriogenesis is introduced into ischemic tissue by ex vivo method. The ex vivo method is defined to be self-transplantation of host cells transfected with a certain gene. In this study, primary cultured skin fibroblasts were derived from host rabbit, target animal for gene incorporation, and used for infection. Then gene transfected fibroblasts were administrated by intraarterial catheters

The second feature of this invention is that recombinant human bFGF fused with secretory signal sequence is introduced. Native bFGF gene does not contain secretory signal sequence, thus extracellular secretion hardly occurs. In this study, recombinant bFGF gene fused with interleukin-2 (IL-2) secretory signal sequence was utilized. Insertion of the secretory signal sequence enables bFGF secretion from the transfected cells. Moreover, this recombinant bFGF is confirmed to maintain biological activity equivalent to the native bFGF, as well as even more stable at extracellular circumstance. Meanwhile, when a growth factor other than bFGF is utilized, the growth factor may inherently contain a secretory signal sequence. In such case, addition of a secretory signal sequence is not requisite. However, regardless of inserted or inherent, a sequence for secretion should be contained anyway.

The third feature of this invention is that adenovirus vector is adopted for introduction of bFGF gene into host fibroblast. Adenovirus vector can achieve highest efficacy of gene incorporation (100% in vivo) among vectors currently utilized and exhibits very high expression of the target gene. On the other hand, when adenovirus vector is used, gene transfected cells existing in host body are immunologically eliminated within several weeks.

Therefore, taking all above features together, transfected fibroblasts are administrated through catheter into ischemic hind limb via arteries administrating the ischemic region, the transfected fibroblasts are retained at capillary vessels of peripheral arteries and persistent secretion of bFGF occurs in the ischemic tissue, which are technical features of this invention. As infected cells are immunologically eliminated from a living body within a certain period, occurrence of unexpected complications caused by long-period expression of bFGF can be inhibited. From this aspect, this method can be recognized to be an effective and safe gene therapy targeted to angiogenesis and arteriogenesis.

For induction of angiogenesis and arteriogenesis according to this invention, various growth factors can be adopted. In concrete, basic fibroblast growth factor (bFGF) gene, acidic fibroblast growth factor (aFGF) gene, vascular endothelial growth factor gene and hepatocyte growth factor gene can be exemplified as collateral development inducible growth factor genes, which can be utilized by insertion into adenovirus vector. Particularly, bFGF, used in the following example, is the most preferred because bFGF is known to be free from causing progression of retinopathia diabetica.

Furthermore, the method according to this invention is applied to model animals of ischemic heart disease, which result in increased cardiac blood flow and improvement of cardiac function. It is assumed that this phenomenon is caused by increased cardiac flow rate of the ischemic region. Improvement in cardiac function can be recognized from the results of measurement of various parameters. Therefore, the method according to this invention is effective for treatment of ischemic heart disease, and a novel and promising therapeutic method for ischemic heart disease is provided. For injected bFGF secreting fibroblasts are removed from host tissues within several weeks, side-effects caused by unnecessary long period of bFGF secretion are not likely to occur. Therefore, a method for treatment of ischemic heart disease with high safety can be provided according to this invention. Moreover, fibroblasts are transferred through catheter in this system, thus less invasive. Therefore, ex vivo method according to this invention can be easily combined with catheter insertion method, conventionally adopted for treatment of ischemic heart disease.

Incidentally, this method can be applied to various creatures, so long as the creature has developed vascular system. In concrete, this method can be applied to various animals such as rabbit, rat, guinea pig, chimpanzee and monkey, as well as human being. Moreover, any non-hematocyte can be utilized as cell used for ex vivo incorporation in this invention. The non-hematocyte used in this method may preferably be cell constituting vessel walls. More preferably, it may be fibroblast, smooth muscle cell or endothelial cell. Fibroblast, utilized in the following example, may be the most preferred cell, considering convenience of collection and separation.

### EXAMPLES

(In Vitro Study)

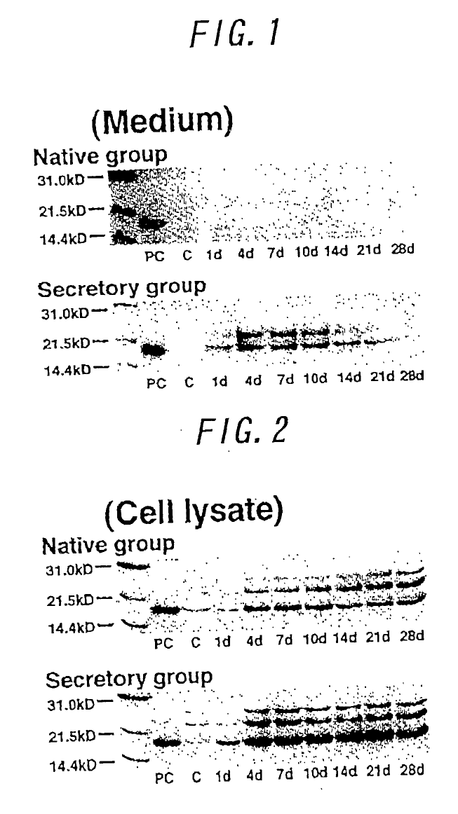

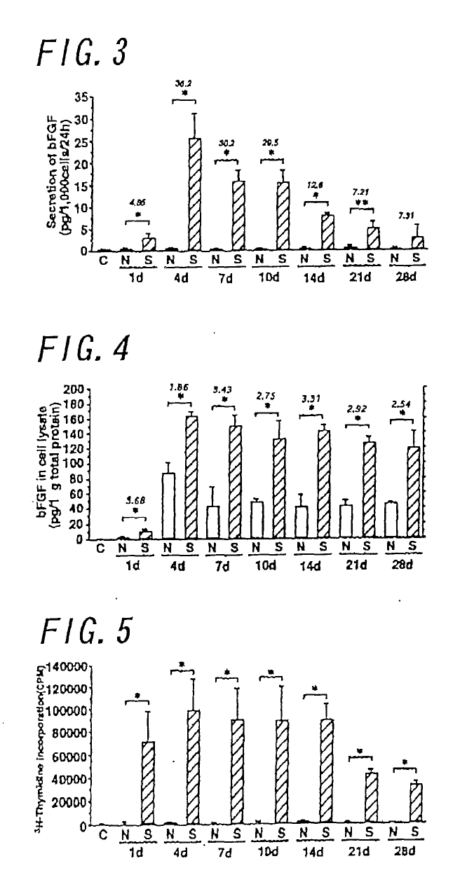

To assess secretion and expression of the infected cells, cultured rabbit fibroblasts were infected with adenovirus vector containing modified human bFGF cDNA with (AxCAMAssbFGF, secretory group) or without the signal sequence (AxCAJSbFGF, native group). Western blot analysis showed the time course of bFGF expression in both the culture medium and the cell lysate. Two forms of bFGF (18 and 22 kD) were observed in the medium of the secretory group with a maximum at 4-10 days after infection, though no bFGF was detected in the native group medium (FIG. 1). Another form (24 kD) of bFGF was detected in the cell lysate (FIG. 2). The enzyme-linked immunosorbent assay (ELISA) data showed that the secreted bFGF level in the medium of the secretory group was significantly higher than that in the native group from 1 day to 21 days after infection, and that the ratio of the secretory group value to the native group value at each time point was 4.85-36.2 (FIG. 3). Although the expressed bFGF in the cell lysate of the secretory group was also significantly higher than that in the native group from day 1 to day 28, the ratio of the secretory group value to the native group value at each time point was 1.86-3.68, which was lower than that of secreted bFGF values in the medium (FIG. 4). The DNA synthesis activity of secreted bFGF in the medium was quantified in cultured fibroblasts by incorporation of ³H-thymidine. The uptake of ³H-thymidine by rabbit fibroblasts increased when the conditioned medium of the secretory group was added, and this uptake was significantly higher than that in the native group until 28 days after infection (FIG. 5).

(Ex Vivo Gene Transfer and Distribution of Administered Cells)

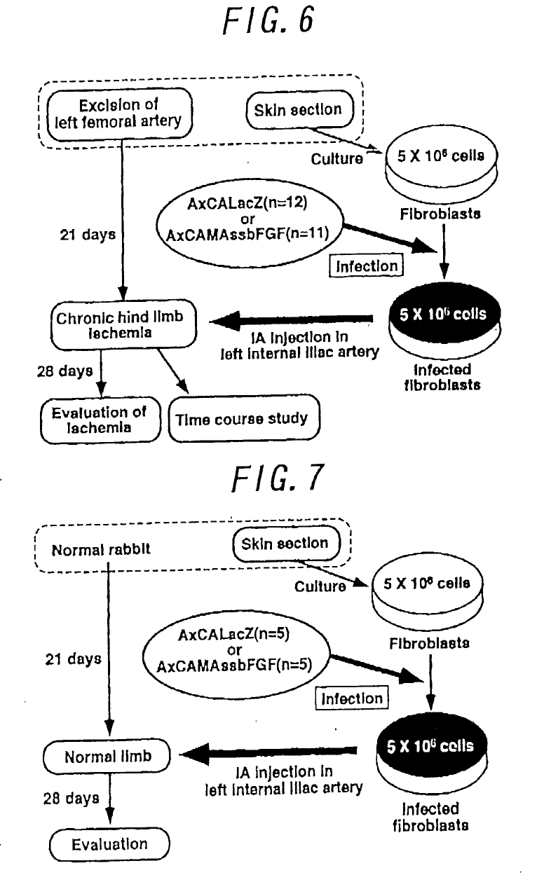

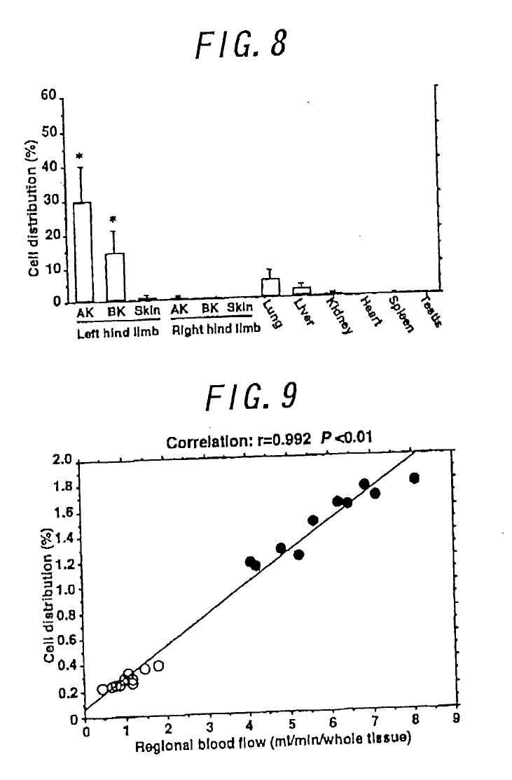

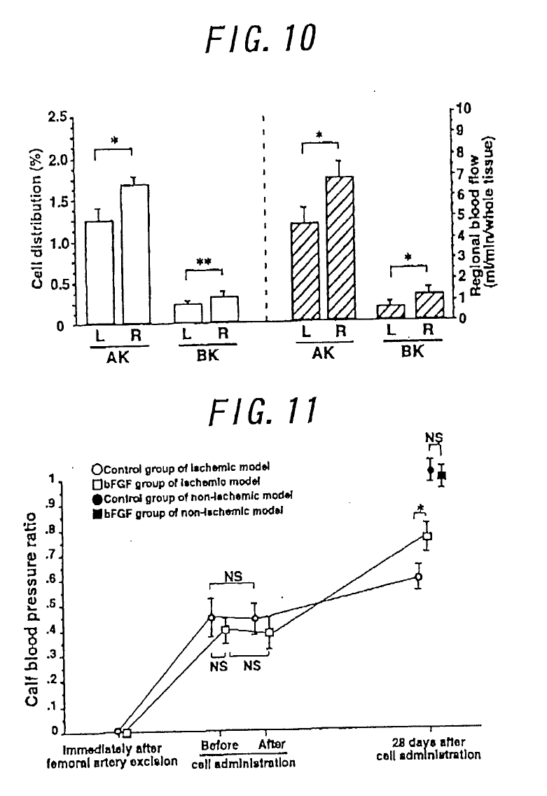

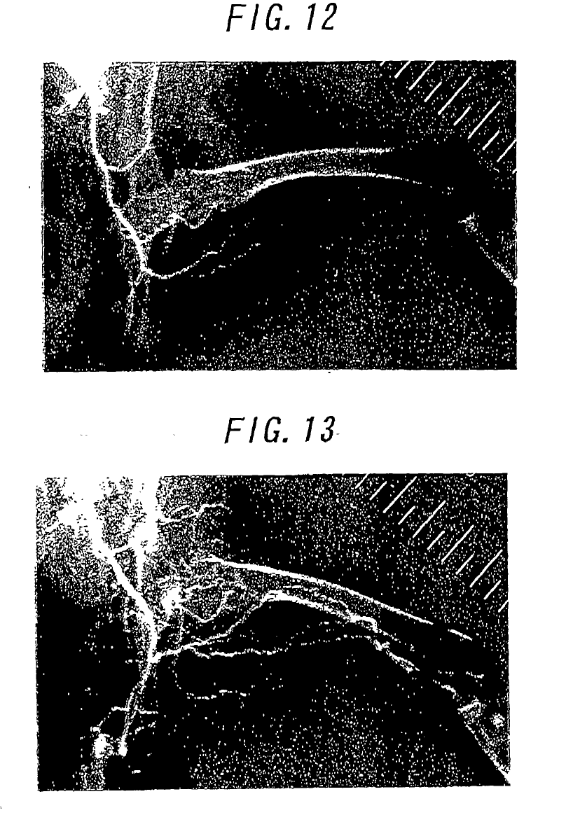

For evaluation of the angiogenic response in vivo, the left femoral artery of the rabbit was completely excised (ischemic model). At 21 days after femoral artery excision, 5×10⁶ fibroblasts, infected with AxCALacZ (control group, n=12) or AxCAMAssbFGF (bFGF group, n=11), were injected as a bolus via the left internal iliac artery (FIG. 6, FIG. 7). Before the experiment, ¹¹¹In-labeled fibroblasts were injected into rabbits in the same manner, and the distribution of administrated cells in organs and tissues was assessed. The distribution of ¹¹¹In-labeled fibroblasts revealed significant accumulation of cells in above- and below-knee muscles of the left hind limb (FIG. 8). Although no significant accumulation was detected in other organs and tissues, 5.4% and 2.7% (mean) of the labeled cells were detected in the lung and liver, respectively.

To determine whether the significant cell accumulation in the left limb was specific to ischemic tissue, we injected both In-labeled fibroblasts and ⁵¹Cr-labeled microspheres into the left ventricle of the same rabbit, and compared the cell distribution with regional blood flow calculated from the microsphere data. Cell distribution in the bilateral hind limbs was highly correlated with their regional blood flow (FIG. 9), and both cell distribution and regional blood flow in muscles of the right hind limb were significantly higher than those of the left ischemic hind limb (FIG. 10).

(Calf Blood Pressure Ratio)

In the study using the ischemic model, the ratio of left calf systolic pressure to right calf systolic pressure (calf blood pressure ratio) showed no significant difference between the control group and bFGF group before cell administration. At 28 days after infected cell injection, calf blood pressure ratio in the bFGF group was significantly higher than that in the control group (FIG. 11). To evaluate the influence of intra-arterial injection of 5×10⁶ fibroblasts, calf blood pressure ratio was measured immediately after injection, and no significant difference was detected between the pressure ratio immediately before and after cell administration. The inventors also examined the effect of collateral development in non-ischemic tissue; 5×10⁶ fibroblasts infected with AxCALacZ (n=5) or AxCAMAssbFGF (n=5) were injected through the left internal iliac artery of normal rabbit (non-ischemic model, FIG. 7). In the non-ischemic model, the ex vivo gene transfer induced no effect on calf blood pressure ratio at 28 days after cell administration (FIG. 11).

(Angiographic Score)

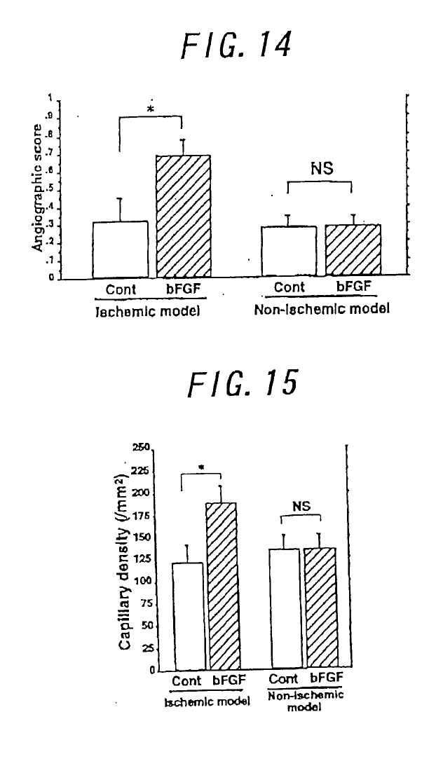

At 28 days after administration of infected fibroblasts to rabbits of the ischemic model, angiographs showed few collateral arteries in the control group (FIG. 12). In contrast, many collateral vessels had developed in the bFGF group (FIG. 13). Angiographic score in the bFGF group demonstrated a significant increase of collateral vessels as compared with that in the control group, while no significant difference was detected in the study using the non-ischemic model (FIG. 14).

(Capillary Density, Arterial Diameter, and In Vivo Blood Flow)

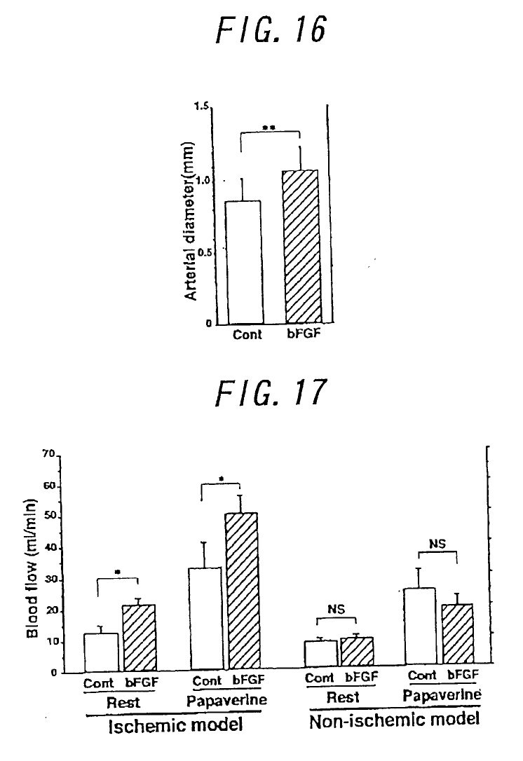

In the bFGF group of the ischemic model, capillary density of the left semimembranous muscle was significantly higher, diameter of the left caudal gluteal artery was significantly larger, and blood flow at rest and maximum blood flow of the left internal iliac artery were also significantly higher than those in the control group (FIGS. 15, 16 and 17). On the contrary, in the non-ischemic model, no significant difference between the two groups was observed in capillary density and blood flow.

(Systemic bFGF Level and Anti-Adenovirus Antibody Titer)

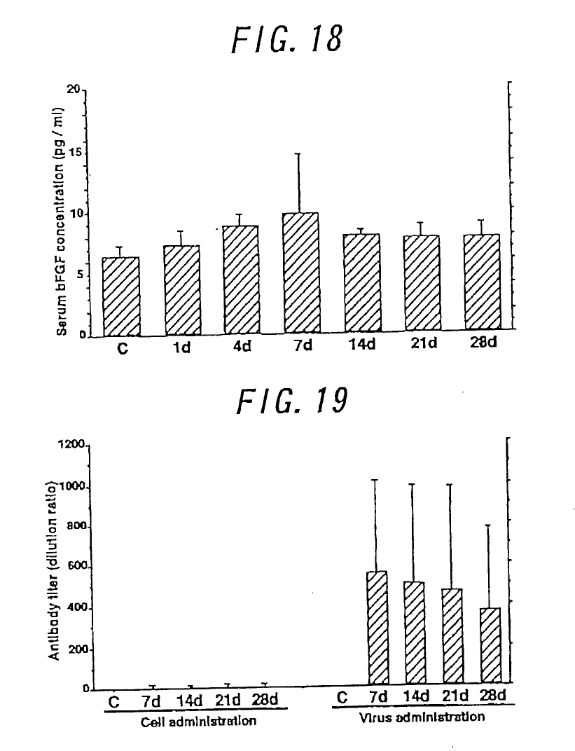

ANOVA analysis detected no significant change in the time course of systemic BFGF level after injection of AxCAMAssbFGF-treated cells into the rabbit ischemic model (FIG. 18). Anti-adenovirus antibody titer was significantly lower in animals with infected cell administration at all time points as compared to that in rabbits with intravenous injection of AxCAMAssbFGF (positive control) (FIG. 19).

(Fate of Gene-Transduced Fibroblasts In Vivo)

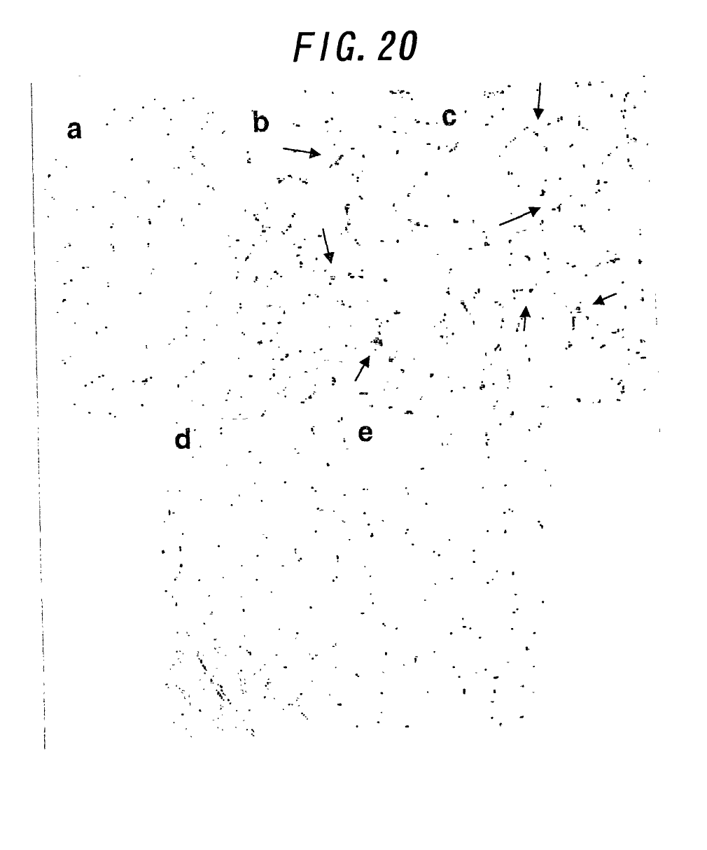

To evaluate the fate of the administrated cells and their influence on host tissues, rabbits of the ischemic model were killed at various time points after injection of AxCAMAssbFGF-treated fibroblasts. Immunostaining for bFGF showed that a large number of bFGF-positive cells were scattered in the left adductor muscle at 1, 4 and 7 days after injection of the infected cells, while bFGF-positive cells were few in control slides (FIG. 20a, FIG. 20b and FIG. 20c). The bFGF-positive cells subsequently decreased, and the number of cells after day 14 was almost equal to that of control (FIG. 20d and FIG. 20e). In internal organs, no significant change in the number of bFGF-positive cells was detected in the time course study. Hematoxylin/eosin (HE) staining and Elastica van Gieson (EVG) staining revealed neither fibrosis nor other changes in all tissues until 28 days after cell administration.

(Local bFGF Accumulation in vivo)

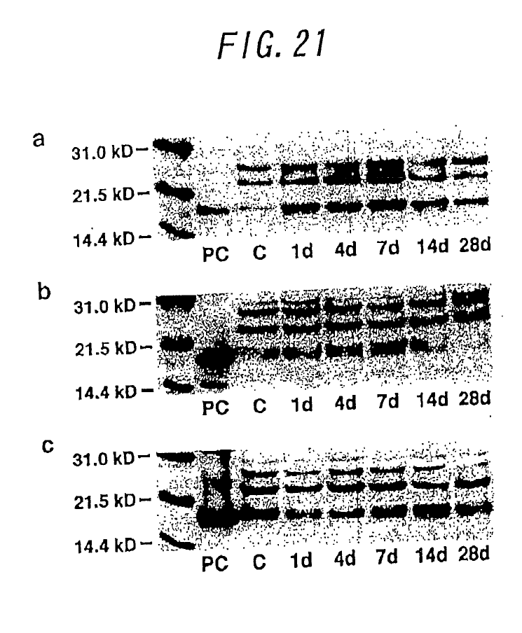

Local accumulation of bFGF in tissues was analyzed by western blot after concentration using heparin-Sepharose. In the left adductor muscle, bFGF accumulation was increased from 1 day after cell injection, and abundant bFGF was observed at day 4 and 7 (FIG. 21a). The protein level decreased after that, though a slightly high amount of bFGF was detected until 28 days after cell administration as compared with that of control. In lung and liver tissues, the time course of bFGF accumulation showed no meaningful increase above the control level (FIGS. 21b and c).

(Calf Blood Pressure Ratio)

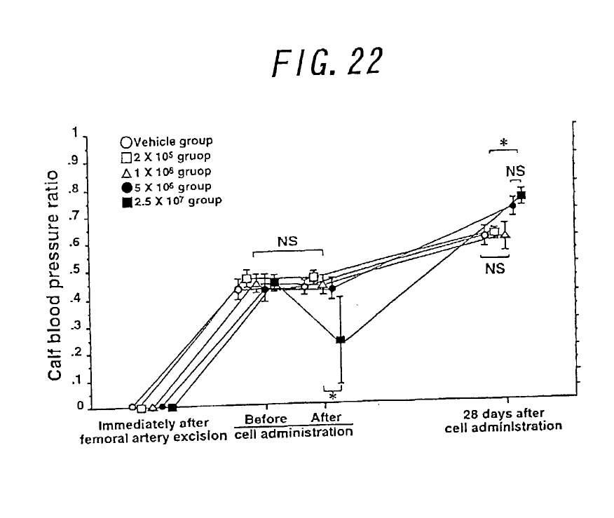

In the present study, four animal groups, in which the numbers of injected fibroblasts were 2×10⁵ (2×10⁵ group), 1×10⁶ (1×10⁶ group), 5×10⁶ (5×10⁶ group) and 2.5×10⁷ (2.5×10⁷ group), and one control group with vehicle injection were examined in a rabbit model of hind limb ischemia. The ratio of left calf systolic pressure to right calf systolic pressure (calf blood pressure ratio) showed no significant difference between all the groups before administration of cells or vehicle. At 28 days after injection, calf blood pressure ratio in the 5×10⁶ group and 2.5×10⁷ group was significantly higher than that in the other three groups, while no significant difference was detected between the 5×10⁶ group and 2.5×10⁷ group, and also between the other three groups. To evaluate the influence of intra-arterial injection of fibroblasts, calf blood pressure ratio was measured immediately after injection, and only the data in the 2.5×10⁷ group were significantly lower than those in the other groups (FIG. 22).

(Angiographic Score)

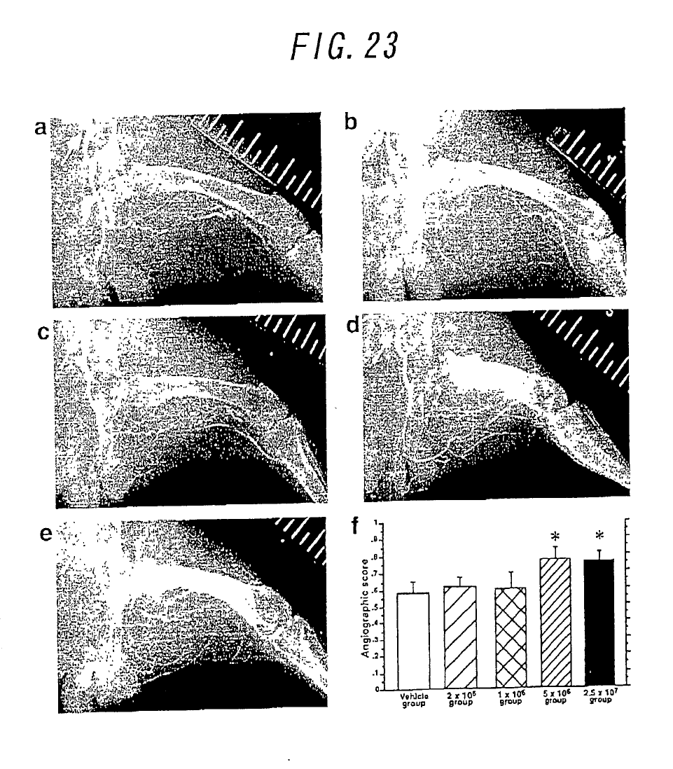

FIG. 23 shows the result of internal iliac angiograms of rabbits, at 28 days after injection of vehicle (a) and 2×1 (b), 1×10⁶ (c), 5×10⁶ (d) and 2.5×10⁶ (e) AxCAMAssbFGF-transduced fibroblasts. Arrow indicates internal iliac artery. Development of collateral vessels was quantified by the angiographic score 28 days after injection (f). Angiograms taken at 28 days after injection showed well-developed collateral vessels in the 5×10⁶ and 2.5×10⁷ groups as compared with the 2×10⁵, 1×10⁶ and vehicle groups (FIGS. 23a, 23b, 23c, 23d and 23e). Angiographic score in the 5×10⁶ and 2.5×10⁷ groups was significantly higher than that in the other three groups (FIG. 23f).

(In Vivo Blood Flow)

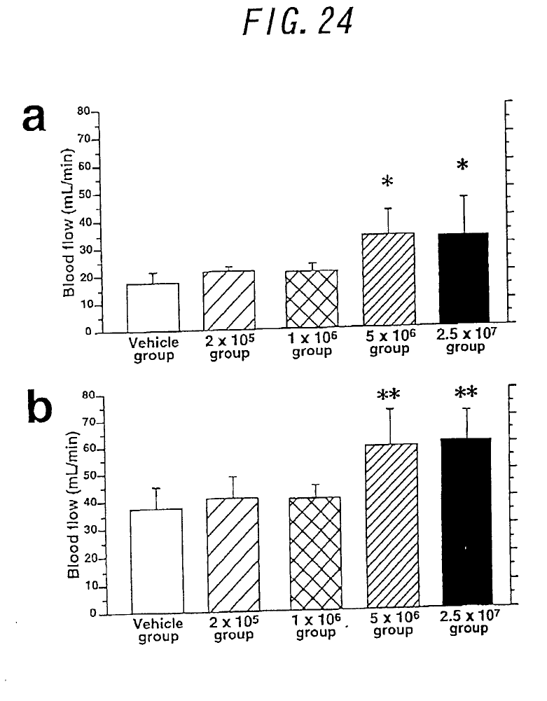

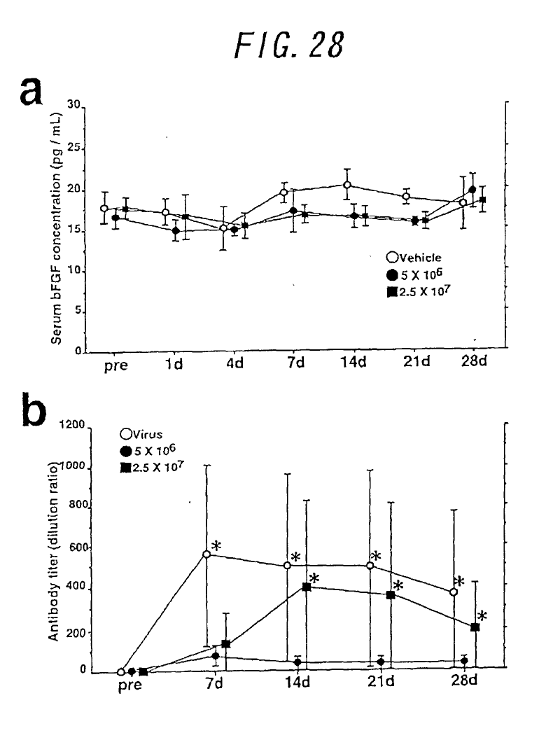

In FIG. 24, in vivo blood flow of left internal iliac artery at rest and maximum in vivo blood flow of left internal iliac artery are shown. In the 5×10⁶ and the 2.5×10⁷ groups, blood flow of the left internal iliac artery at rest was significantly higher than that in the vehicle group (FIG. 28a). In the 5×10⁶ and 2.5×10⁷ groups, maximum blood flow of the left internal iliac artery after papaverine injection was significantly higher than that in the 2×10⁵, 1×10⁶ and vehicle groups (FIG. 24b).

(Capillary Density)

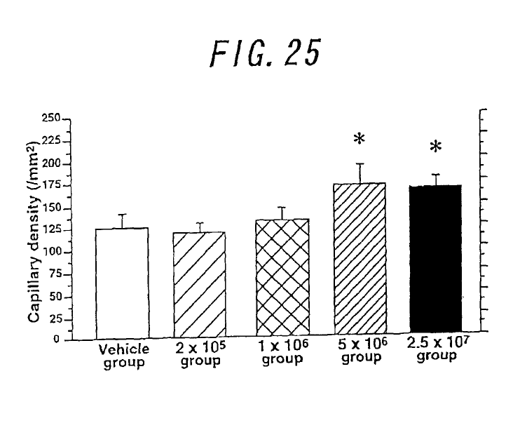

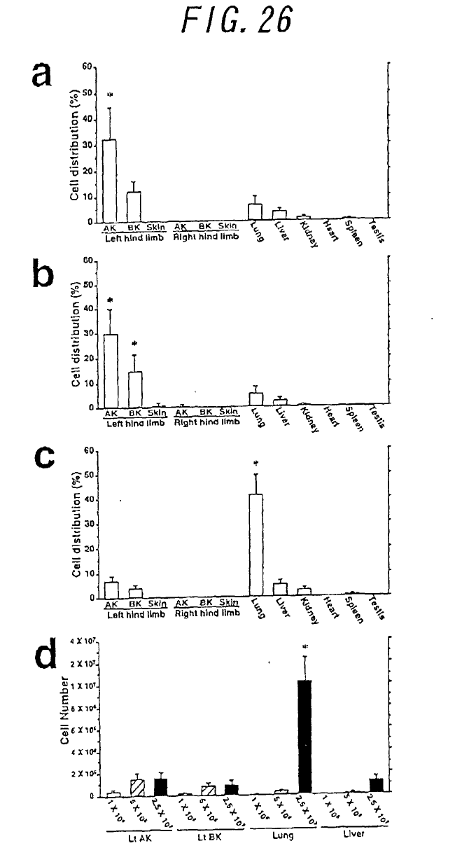

Capillary density of left semimembranous muscles was measured in tissue sections stained by indoxy-tetrazolium method. Capillary density in the 5×10⁶ and 2.5×10⁷ groups was significantly higher than that in the 2×10⁵, 1×10⁶ and vehicle groups (FIG. 25).

(Distribution of Administered Cells)

To evaluate the distribution of injected cells, ¹¹¹In-labeled fibroblasts were administered into the left internal iliac artery of rabbits with hind limb ischemia. Three animal groups injected with 1×10⁶, 5×10⁶ and 2.5×10⁷ cells were analyzed. Cell numbers of ¹¹¹In-labeled fibroblasts in above- (AK) and below-knee (BK) muscles of the left hind limb, lung, and liver at 5 hours after injection of 1×10⁶, 5×10⁶, and 2.5×10⁷ cells into the left internal iliac artery are shown. In FIG. 26, 1×10⁶ cells (a), 5×10⁶ cells (b), and 2.5×10⁷ cells (c) are injected into the left internal iliac artery, distribution of ¹¹¹In-labeled fibroblasts in organs and tissues at 5 hours after injection and data presented as percentage of radioactivity distributed in each tissue relative to total radioactivity of administered cells (d) are shown. Distribution data (%) showed significant accumulation of labeled cells in the above- (AK) and below-knee (BK) muscles of the left hind limb in animals treated with 1×10⁶ and 5×10⁶ cells (FIGS. 26a and 26b). Only in rabbits treated with 2.5×10⁷ cells, significant accumulation was observed in lung (FIG. 26c). Although the distribution (%) in the above- and below-knee muscles of the left hind limbs differed according to the number of injected cells, there was no significant difference in accumulated cell number between the animals treated with 5×10⁶ cells and 2.5×10⁷ cells (FIG. 26d).

(In Vivo Expression of bFGF Protein)

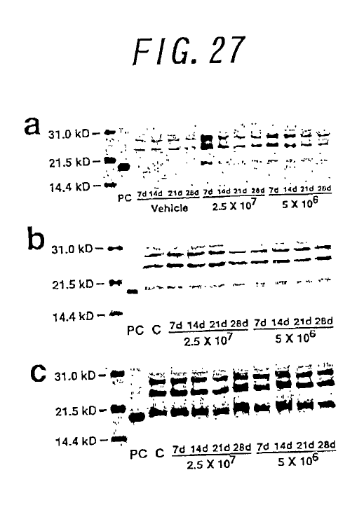

Western blot after concentration using heparin-sepharose showed local accumulation of expressed bFGF. In FIG. 27, time course of bFGF accumulation in left adductor muscle (a), lung (b) and liver (c) are shown. PC indicates positive control and Vehicle indicates vehicle-treated control sample. In vehicle-treated control rabbits, the time course of bFGF accumulation in the left adductor muscle revealed no meaningful change (FIG. 27a). In rabbits treated with 5×10⁶ cells and 2.5×10⁷ cells, bFGF accumulation in the left adductor muscle was increased at 7 and 14 days after cell administration as compared with animals with vehicle injection. bFGF accumulation decreased thereafter, though the amount of bFGF on day 21 and 28 was slightly higher than that in vehicle-treated control rabbits. At 7 days after cell injection, bFGF accumulation in the adductor muscle was increased in rabbits treated with 2.5×10⁷ cells as compared with those treated with 5×10⁶ cells, but on days 14, 21, and 28, no distinct difference in bFGF accumulation was detected between the two groups (FIG. 27a). In lung and liver tissue, the time course of bFGF level revealed no meaningful change from the control level (FIGS. 27b and 27C).

(Systemic bFGF Level and Anti-Adenovirus Antibody Titer)

In FIG. 28, time course of systemic bFGF level measured by ELISA (a) and time course of anti-adenovirus antibody titer quantified by neutralizing test (b) are shown. Titers are shown as dilution ratio, and titers less than 1:4 were assigned a value of 1. Time course analysis of systemic bFGF level indicated no significant increase of bFGF in rabbits administered with 5×10⁶ cells, 2.5×10⁷ cells and vehicle (FIG. 28a). Anti-adenovirus antibody titer was significantly lower in animals injected with 5×10⁶ cells as compared with that in rabbits with intravenous injection of AxCAMAssbFGF (positive control) (p<0.05), and the titer in animals treated with 2.5×10⁷ cells was significantly higher than that with 5×10⁶ cells on days 14, 21 and 28 (FIG. 28b).

(Side-Effects After Administration of Gene-Transduced Fibroblasts)

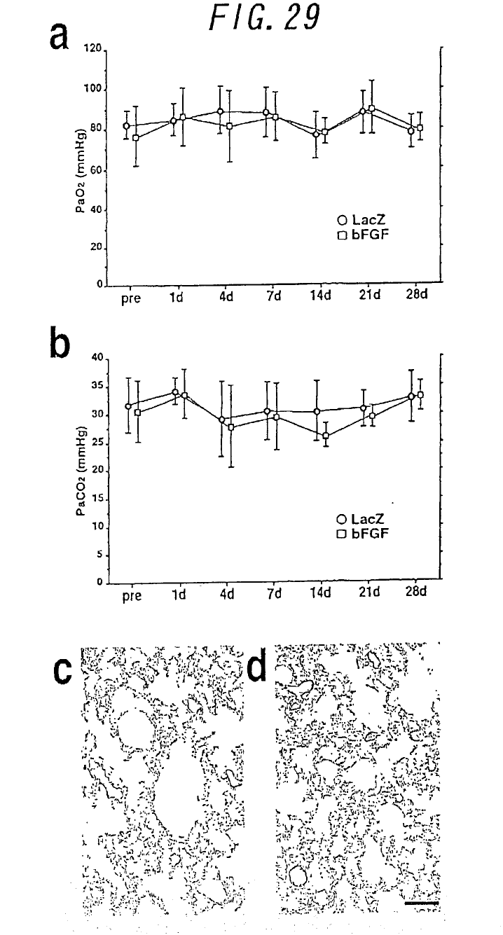

Blood analysis and histological evaluation of the time course samples were carried out to determine the side-effects caused by injection of gene-transduced cells. In FIG. 29, time course of PaO₂ (a) and PaCO₂ (b) in blood gas analysis and microphotograph of lung with Elastica van Gieson staining in AxCAMAssbFGF-treated (c) and AxCALacZ-treated (d) rabbits are shown. Both complete blood count and blood chemical tests showed no abnormal data in animals treated with 5×10⁶ cells and 2.5×10⁷ cells. Further, histological studies using hematoxylin/eosin and Elastica van Gieson staining revealed no fibrosis or other abnormal changes in all tissues by day 28.

(Intravenous Injection of Gene-Transduced Fibroblasts)

After the administration of gene-transduced cells via the left internal iliac artery, other than in the left hind limb, the cells were predominantly distributed in the lungs, suggesting that cells not trapped in the left hind limb tissues entered the venous system and then accumulated in lung tissue. To assess the influence of such cells, we administered AxCAMAssbFGF-treated fibroblasts through the iliac vein of normal rabbits. Blood gas analysis showed no significant changes in PaO₂, PaCO₂ and other parameters throughout the time course and also as compared with control (FIGS. 29a and 29b). Further, histological studies also showed no abnormal findings such as fibrosis as compared with control until 28 days after the injection (FIGS. 29c and 29d).

In the above-described experiments, using rabbit as model animal, administrated cell number was optimized to avoid occurrence of side-effects. Then, the rabbit showed no significant increase of collateral development in 2×10⁵ cells injected group (2×10⁵ group) and in 1×10⁶ cells injected group (1×10⁶ group). Meanwhile, well-developed collateral vessels were observed in 5×10⁶ cells injected group (5×10⁶ group). Therefore, to obtain desired effects in this model, is was estimated that injection of more than 1×10⁶ cells to 5×10⁶ cells was requisite.

Moreover, when this model was utilized, no significant difference in collateral augmentation was observed between 2.5×10⁷ cells injected group (2.5×10⁷ group) and the 5×10⁶ group. One possible explanation of this phenomenon is the capacity of the hind limb muscles to retain the cells in their capillaries and small arteries. At 5 hours after injection of labeled fibroblasts, accumulated cell number in the left hind limb muscles showed no significant difference between the 5×10⁶ group and the 2.5×10⁷ group, while the cell distribution data revealed markedly greater cell accumulation in the lungs of rabbits of 2.5×10⁷ group than in other animals. These findings suggested that surplus cells exceeding the capacity of the tissue overflowed into the venous system and were then trapped in capillaries and small arteries of the lung, increasing the possibility of unexpected pulmonary side-effects. Western blot analysis using the left adductor muscle samples showed no remarkable difference between 5×10⁶ group and 2.5×10⁷ group, supporting the above concept. Therefore, to perform this method of this invention in an animal, sufficient number of cells is needed to obtain the effect, but selected condition of cell number should not be too large, in the aspect to avoid occurrence of pulmonary side-effects.

Moreover, administration of 2.5×10⁷ cells significantly decreased calf blood pressure ratio immediately after injection. In contrast, no significant decrease of calf blood pressure ratio was detected after injection of 5×10⁶ cells or fewer. These findings showed that the excessive fibroblasts behaved like emboli in the capillaries and small arteries and reduced peripheral blood flow immediately after injection. By 5 hours after injection, the excessive cells overflowed into the venous system, and then the cell number accumulated in the left hind limb muscles did not exceed a certain limit, as mentioned previously. Therefore, it is believed that the decrease of pressure ratio after injection of 2.5×10⁷ cells was transient. However, the transient drop of the blood pressure potently induces some damages in the ischemic tissues.

Additionally, the serum anti-adenovirus antibody level in rabbits treated with 2.5×10⁷ cells was significantly higher than that in animals treated with 5×10⁶ cells or fewer, indicating that the host was contaminated with viral particles. This might be because three washes after viral infection was insufficient to remove viral particles from 2.5×10⁷ cells. Since the adenovirus vector used in this procedure is replication-deficient, contamination with viral vector does not induce severe side-effects; however, the possibility that replication competent virus may appear, must be considered.

(In Vitro Study)

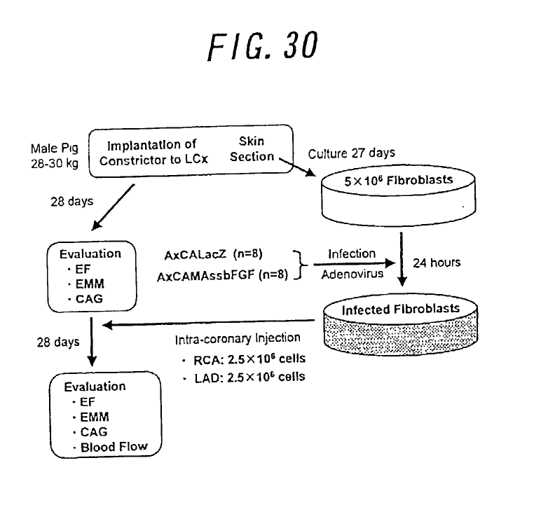

Furthermore, using an animal model of ischemic heart disease, the inventors investigated on whether the method according to this invention is effective for treatment of ischemic heart disease or not. Schematic figure of experimental protocol is shown in FIG. 30. Cultured pig fibroblasts confluent in 60 mm dishes (passage 3) were infected with AxCAMAssbFGF at 20 p.f.u/cell in 1 mL of Dulbecco's modified Eagle's minimum medium (DMEM, Gibco BRL, NY) with 2% FBS (DMEM-2%). After 1-hour incubation, the infected fibroblasts were washed twice with PBS, and then cultured in 3 mL DMEM-2%. The medium was changed daily and stored at 1, 4, 7, 10, 14, 21 and 28 days after infection. The inventors excluded samples contaminated with residual virus by applying them to 293 cells and observing them for 14 days. Another set of pig fibroblasts was cultured in DMEM-2%, and daily-changed culture medium was used as control. Each medium (50 μL) was subjected to Western blot analysis using mouse monoclonal antibody against bovine bFGF (1:500, Upstate Biotechnology), and the DNA synthesis activity of secreted bFGF in the culture medium (100 μL) was evaluated by incorporation of ³H-thymidine into pig fibroblasts. These in vitro analyses were repeated at least twice.

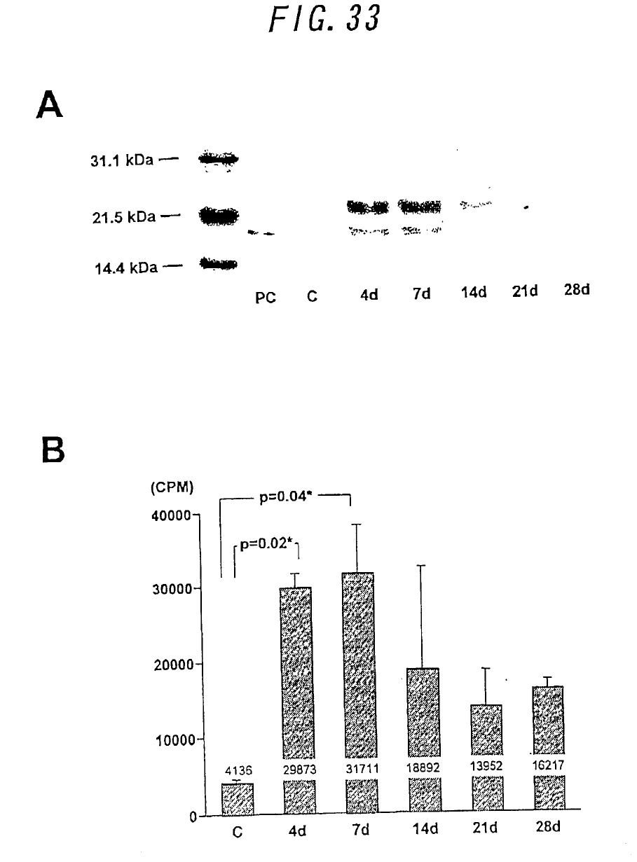

Western blot analysis showed that bFGF protein was secreted in the culture medium of AxCAMAssbFGF-treated pig fibroblasts. Two forms of bFGF (18 and 22 kDa) were detected in the medium with a maximum at 4-10 days after infection (FIG. 33a). By ³H-thymidine incorporation assay, at 4 and 7 days after infection, the DNA synthesis activity of the bFGF was demonstrated to be higher as compared with the control (FIG. 33b).

(Animal Model of Chronic Myocardial Ischemia)

The inventors used a pig model of chronic myocardial ischemia induced with ameroid constrictor for in vivo evaluation. Male LWD pigs (Saitama Experimental Animals Supply, Saitama, Japan) weighing 28-30 kg were anesthetized with ketamine hydrochloride (15 mg/kg, IM), pentobarbital sodium (10 mg/kg, IV), and vecuronium bromide (2 mg, IV), intubated, and ventilated with room air. Pentobarbital was added for maintaining adequate anesthesia. Intra-arterial blood pressure and a limb lead electrocardiogram were monitored, and both ampicillin (500 mg, IM) and lidocaine (30 mg, IM) were administrated prior to surgical procedure. After a left thoracotomy, a metal-encased ameroid constrictor with 2.5 mm lumen (Research Instruments SW, CA, USA) was placed around the proximal left circumflex branch (LCx). Preliminary experiments revealed constrictors of this size occluded the LCx within 28 days. Simultaneously, 10×10 mm section of skin was obtained for fibroblast culture.

(Ex Vivo Gene Transfer)

Fibroblasts were cultured from the resected skin to confluent in 100-mm dishes (3 passages). At 27 days after constrictor implantation (Day 27), 5×10⁶ fibroblasts were infected with AxCAMAssbFGF (bFGF fibroblast) or AxCALacZ (LacZ fibroblast) at 20 p.f.u/cell and incubated for 24 hours. At Day 28, under systemic heparinization (2,000U), a 6-French guiding catheter (Britetip JL4, Cordis Endovascular Systems, FL, USA) was inserted to the right coronary artery (RCA) via the right common carotid artery, and a thin infusion catheter (Transit 2, Cordis Endovascular Systems) was introduced through the guiding catheter into the RCA and positioned at 20 mm distal to the orifice. Subsequently, 2.5×10⁶ bFGF-fibroblasts (n=8) or LacZ fibroblast (n=8) suspended in 5 mL of DMEM-2% were injected through the infusion catheter. Remaining 2.5×10⁶ infected fibroblasts of each group were injected into the left anterior descending artery (LAD) in the same manner (FIG. 30). Plasma cardiac troponin-I was measured immediately before, 12 hours and 24 hours after fibroblast injection to detect myocardial infarction during these procedures.

(Influence of Intra-Coronary Cell Administration)

Although ST-T changes in the electrocardiogram were observed in 6 (37.5%) pigs during or immediately after intra-coronary fibroblast injection, these changes were recovered within 5 minutes. In addition, values of plasma cardiac troponin-I were all under the lower limit of detection (<0.3 ng/mL), suggesting no significant influence of micro-embolism.

(Echocardiography)

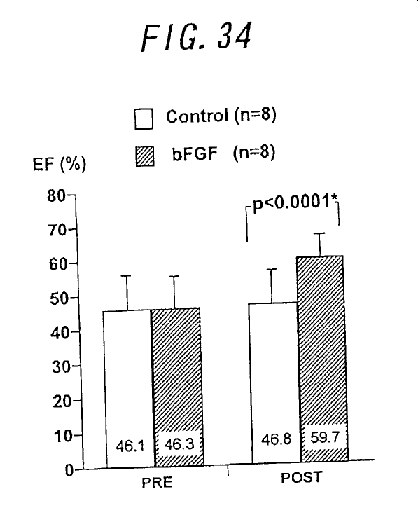

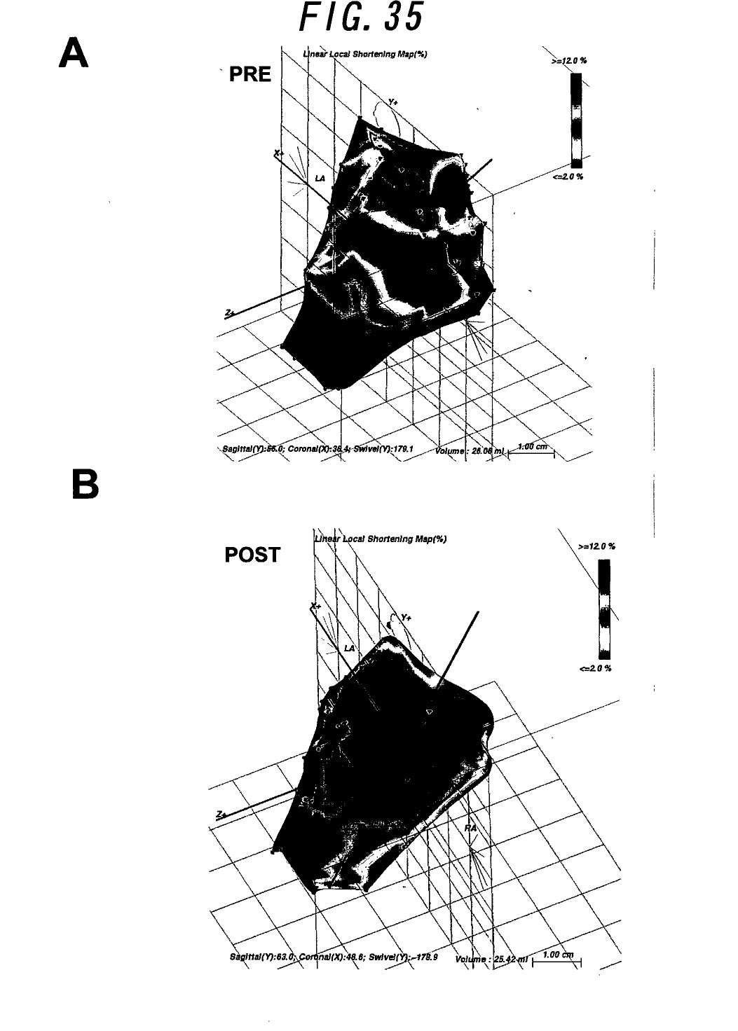

Trans-thoracic echocardiography was conducted immediately before and 28 days after fibroblast administration. Ejection fraction (EF) of left ventricle (LV) is shown in FIG. 34. Ejection fraction (EF) was measured from a short axis view of the left ventricle (LV) at the level of the papillary muscles. As a result, the bFGF group showed significantly greater improvement of the EF as than the control group (FIG. 34).

(Electromechanical Mapping: EMM)

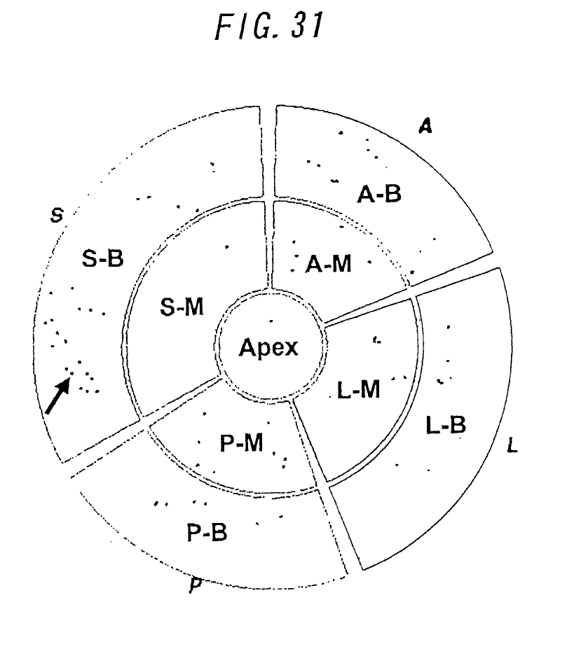

EMM was performed using the NOGA system (Version 4.0, Biosense, Israel), which was described previously, immediately before and 28 days after injection of fibroblasts. Briefly, a mapping catheter (NOGA-STAR, B-curve, Biosense-Webster, CA, USA) was inserted into the LV, the data of the endocardial movements and electrograms were collected from more than 40 sites, and a 3-dimensional endocardial local shortening (LS) or a unipolar endocardial voltage (UpV) map was constructed. LS represents myocardial mechanical function, and UpV represents myocardial viability. For the analysis of the data, the constructed LV map was divided into 3 segments by 2 planes vertical to the LV long axis; apex, midventicle and base. Each segment contained 20, 40 and 40% of the length of the LV long axis, respectively. Then, each of the latter 2 segments was divided into 4 regions; anterior, septal, posterior and lateral. Namely, the LV was divided into 9 regions. This division was performed semi-automatically by the NOGA computer system, and the results are expressed as a Bull's eye-like diagram (FIG. 31). In FIG. 31, each point (arrow) shows the data-sampling points. A represents anterior, S represents septal, M represents midventicle, and B represents base.

Fisher's combination of p-values in the 9 sets of LS data was 37.1[>χ2(18,0.05)=34.8], indicating significant global difference in the improvement of LS between the bFGF and control groups. Following regional analysis showed that the bFGF group revealed significant greater improvement of LS in the posterior 2 segments and the lateral-midventicle segment than the control group (Table 1, FIG. 35). Contrarily, the UpV data showed no significant difference between the bFGF and control groups (Table 1). Table 1 shows the results of electromechanical mapping, which represent LS (%) data and UpV (mV) data obtained at pre-cell injection (PRE) and post-cell injection (POST), respectively. FIG. 35 shows three-dimensional local shortening (LS) maps (a) before administration of fibroblasts and (b) 28 days after the treatment, and the postero-lateral region, facing the front, exhibits (a) decreased (red, yellow, or green) and (b) improved (blue or purple) LS, respectively.

TABLE 1

| | | | LS (%) | | | UpV (mV) | |
|---|---|---|---|---|---|---|---|
| Area | Group | PRE | POST | p-value | PRE | POST | p-value |
| Apex | bFGF | 13.7 ± 6.1 | 13.0 ± 4.6 | 0.74 | 2.44 ± 0.91 | 2.69 ± 1.25 | 0.45 |
| | Control | 13.1 ± 6.4 | 11.0 ± 4.0 | | 2.45 ± 0.78 | 2.21 ± 1.06 | |
| A-M | bFGF | 10.6 ± 6.4 | 9.2 ± 4.1 | 0.57 | 2.09 ± 0.58 | 2.13 ± 1.02 | 0.21 |
| | Control | 13.1 ± 5.6 | 9.3 ± 6.2 | | 2.14 ± 0.83 | 1.68 ± 0.51 | |
| A-B | bFGF | 7.7 ± 4.1 | 6.1 ± 5.2 | 0.45 | 1.34 ± 0.72 | 1.40 ± 0.76 | 0.78 |
| | Control | 9.4 ± 7.9 | 12.4 ± 10.6 | | 1.36 ± 0.80 | 1.58 ± 0.90 | |
| S-M | bFGF | 11.7 ± 5.8 | 14.8 ± 5.6 | 0.70 | 2.06 ± 0.81 | 2.20 ± 0.87 | 0.74 |
| | Control | 12.1 ± 6.8 | 14.0 ± 6.9 | | 1.96 ± 0.85 | 1.96 ± 0.48 | |
| S-B | bFGF | 8.4 ± 5.2 | 11.9 ± 6.0 | 0.73 | 1.69 ± 0.69 | 1.66 ± 0.66 | 0.19 |
| | Control | 9.9 ± 5.9 | 12.3 ± 6.6 | | 1.44 ± 0.56 | 1.95 ± 0.70 | |
| P-M | bFGF | 7.5 ± 5.6 | 13.4 ± 7.8 | 0.043* | 1.98 ± 0.53 | 2.61 ± 0.51 | 0.46 |
| | Control | 7.5 ± 4.3 | 3.8 ± 4.3 | | 1.80 ± 0.82 | 2.11 ± 0.72 | |
| P-B | bFGF | 8.5 ± 6.3 | 12.3 ± 6.3 | <0.0001* | 1.74 ± 0.76 | 2.60 ± 0.81 | 0.10 |
| | Control | 11.3 ± 3.6 | 8.5 ± 4.0 | | 1.51 ± 0.84 | 1.56 ± 0.53 | |
| L-M | bFGF | 9.5 ± 7.5 | 15.5 ± 6.9 | 0.026* | 2.04 ± 1.41 | 2.68 ± 1.34 | 0.26 |
| | Control | 10.0 ± 9.0 | 4.5 ± 4.0 | | 1.51 ± 0.97 | 1.35 ± 0.84 | |
| L-B | bFGF | 9.2 ± 5.2 | 12.3 ± 3.1 | 0.82 | 1.38 ± 0.59 | 1.65 ± 0.56 | 0.29 |
| | Control | 7.8 ± 10.9 | 9.7 ± 6.5 | | 1.11 ± 0.67 | 1.08 ± 0.40 | |

LS, local shortening;
UpV, inpolar voltage;
A, anterior;
S, septal;
P, posterior;
L, laterial;
M, midventricle;
B, base.

(Coronary Arteriography: CAG)

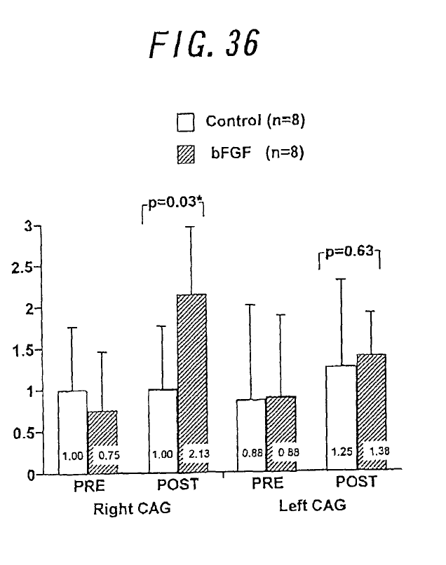

CAG was also conducted immediately before and 28 days after cell administration. First, a 6-French catheter (Britetip JL4) was inserted into the RCA, and 4.5 mL of contrast medium (Iopamiron 370, Schering, Berlin, Germany) was injected at a rate of 1.5 mL/second. Digitally subtracted images were obtained at a rate of 8 frames/second using a C-arm digital fluoroscopy system (Sirus Power/C, Hitachi Medico, Tokyo, Japan) under 2 different angulations, namely, left anterior oblique 20° and right anterior oblique 20° (right CAG). The same procedures were repeated for the left coronary artery (left CAG). For quantitative analysis of the development of the collateral circulation to the LCx, Rentrop scores were obtained from each shot. FIG. 36 shows the Rentrop scores of the bilateral coronary arteriography (CAG).

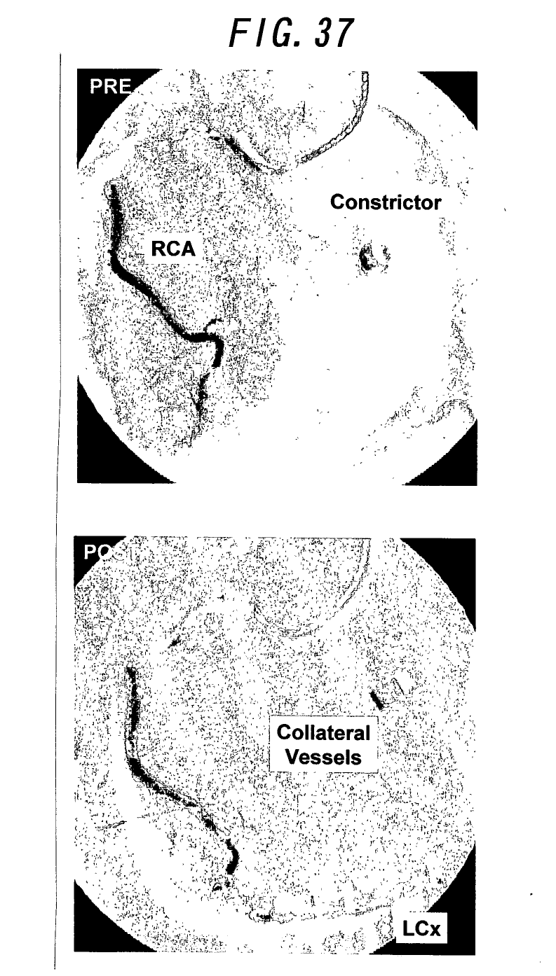

The bFGF group revealed significantly greater improvement in the Rentrop score of the right CAG than the control group (FIG. 36). Meanwhile, no significant improvement was detected in the left CAG. Moreover, the right CAG in a pig belonging to bFGF group obtained (PRE) before and (POST) 28 days after fibroblast injection is shown (FIG. 37). Although the occluded left circumflex branch (LCx) was not enhanced before fibroblast injection (FIG. 37 PRE, Rentrop score=0), several collateral vessels from the right coronary artery (RCA) and partial enehncement of the LCx were observed after fibroblast injection (FIG. 37 POST, Rentrop score=2).

(Regional Myocardial Blood Flow Measurement)

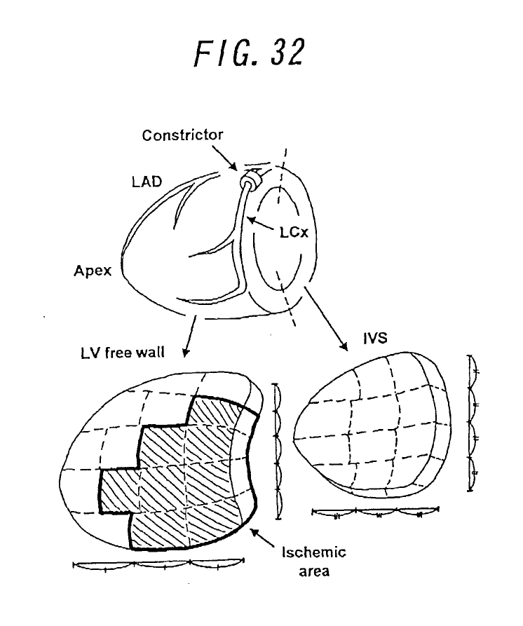

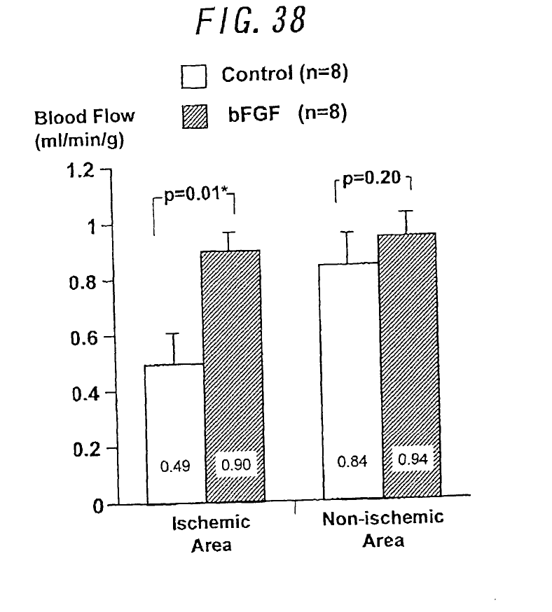

At 28 days after fibroblast injection, 7.5×10⁶ of dye-extraction microspheres were injected into the left atrium after EMM and CAG, and regional myocardial blood flow (RMBF) was measured. A reference blood withdrawn was started 10 seconds prior to microsphere injection and continued for 120 seconds at a rate of 2.5 mL/minute. The LV myocardium was divided into 28 samples as shown in FIG. 32, and weighed (W_{SAMPLE}). Each sample and reference blood was digested with KOH and filtered with 10 μm pore filter to recover microspheres. Dye was extracted from the microspheres, and the absorbance at 448 nm was measured using a spectrophotometer. Average blood flow rate in each of the ischemic and non-ischemic area, which was defined in the LV, was then calculated as (withdrawal rate)×[(ΣA_{SAMPLE}/A_{BLOOD})×(ΣW_{SAMPLE})⁻1 in the respective area, where A_{SAMPLE} was the absorbance of the myocardial samples and A_{BLOOD} was that of the reference blood. FIG. 38 shows myocardial blood flow rate in the ischemic and non-ischemic areas 28 days after fibroblast injection. In the ischemic area, the bFGF group revealed significantly higher blood flow rate than the control group, while no significant difference was observed in the non-ischemic area.

(Distribution of Injected Fibroblasts)

The efficacy of fibroblasts accumulation and distribution of the accumulated fibroblasts in the LV myocardium were assessed. Fibroblasts (6.0×10⁶) infected with AxCAluc+ at 20 pfu/cell and incubated for 24 hours were suspended in 12 mL of DMEM-2%, and mL of this suspension containing 2.5×10⁶ fibroblasts was administrated into each of the RCA and LAD (10 mL in total) of pigs (n=3) implanted with constrictor 28 days before. The amount of luciferase in the remaining suspension (2 mL) was quantified using Luciferase Assay Kit (Promega, WI, USA), and the amount of the whole injected luciferase was calculated (W_{SAMPLE}). Two hour later, the pigs were killed, and LV myocardium was divided into 28 samples (FIG. 32). FIG. 32 shows division of the left ventricle (LV) and definition of the ischemic area in the experiment of regional myocardial blood flow measurement. The LV was divided to the free wall and the septum (IVS), and the former was further divided into 16 fragments and the latter 12. Considering a typical perfusion area of the left circumflex branch (LCx), the area with slant lines, consisted with most part of the postero-lateral wall, was defined as the ischemic area, and the rest was defined as the non-ischemic area. Each sample was weighed, and luciferase in each sample was quantified (L_{SAMPLE}). The percentage of the fibroblasts trapped in the LV myocardium was calculated as 100×Σ(L_{SAMPLE})×(L_{WHOLE})⁻¹. On the other hand, the concentration of the injected fibroblasts in each of the ischemic and non-ischemic area (FIG. 32) was calculated as 5.0×10⁶×(L_{WHOLE})⁻¹×Σ(L_{SAMPLE})×[Σ(L_{SAMPLE})]⁻¹ in the respective area. In the non-ischemic area, significantly higher concentration of fibroblast accumulation was detected compared with that in the ischemic area [(6.9±1.2)×10⁴ versus (2.0±0.8)×10⁴ cells/1 g, p=0.004].

This invention provided a novel method for promotion of angiogenesis and arteriogenesis, wherein a growth factor gene was introduced into fibroblasts ex-vivo using adenovirus vector. Moreover, the method according to this invention could improve cardiac blood flow rate of ischemic region, thereby a novel method for treatment of ischemic heart disease was also provided.

1. A method for promotion of angiogenesis and arteriogenesis, the method comprises the steps of;
(1) preparing modified adenovirus vector by incorporating growth factor gene fused with secretory signal sequence into an adenovirus vector,
(2) obtaining non-hematocytes from a creature being target of the angiogenesis and arteriogenesis, and culturing said non-hematocytes ex-vivo,
(3) infecting cultured said non-hematocytes with said modified adenovirus vector to prepare non-hematocytes having growth factor secretory ability, by introducing said modified adnovirus vector into said non-hematocytes; and
(4) administrating said non-hematocytes having growth factor secretory ability via blood vessel of said creature, thereby said growth factor is secreted in body of said creature.

2. The method according to claim 1, wherein said growth factor gene is selected from the group consisting of basic fibroblast growth factor (bFGF) gene, acidic fibroblast growth factor (aFGF) gene, vascular endothelial growth factor gene and hepatocyte growth factor gene.

3. The method according to claim 1, wherein said secretory signal sequence is secretory signal derived from interleukin-2.

4. The method according to claim 1, wherein said non-hematocyte cells having growth factor secretory ability is administrated into vessel of said creature through a catheter.

5. A method for treatment of ischemic heart disease, the method comprises the steps of;
(1) preparing modified adenovirus vector by incorporating growth factor gene fused with secretory signal sequence into an adenovirus vector,
(2) obtaining non-hematocytes from a creature being target of the treatment of ischemic heart disease, and culturing said non-hematocytes ex-vivo,
(3) infecting cultured said non-hematocytes with said modified adenovirus vector to prepare non-hematocytes having growth factor secretory ability, by introducing said modified adnovirus vector into said non-hematocytes; and
(4) administrating said non-hematocytes having growth factor secretory ability via blood vessel of said creature, thereby said growth factor is secreted in body of said creature.

6. A method to increase cardiac blood flow rate in a creature under myocardial ischemia, the method comprises the steps of;
(1) preparing modified adenovirus vector by incorporating growth factor gene fused with secretory signal sequence into an adenovirus vector,
(2) obtaining non-hematocytes from the creature under myocardial ischemia, and culturing said non-hematocytes ex-vivo,
(3) infecting cultured said non-hematocytes with said modified adenovirus vector to prepare non-hematocytes having growth factor secretory ability, by introducing said modified adnovirus vector into said non-hematocytes; and
(4) administrating said non-hematocytes having growth factor secretory ability via blood vessel of said creature, thereby said growth factor is secreted in body of said creature.

7. Non-hematocytes having growth factor secretory ability, the non-hematocytes produced by the steps of;
(1) preparing modified adenovirus vector by incorporating growth factor gene fused with secretory signal sequence into an adenovirus vector,
(2) obtaining non-hematocytes from a creature, and culturing said non-hematocytes ex-vivo; and
(3) infecting cultured said non-hematocytes with said modified adenovirus vector to prepare non-hematocytes having growth factor secretory ability, by introducing said modified adnovirus vector into said non-hematocytes.
(3) infecting cultured said non-hematocytes with said modified adenovirus vector to prepare non-hematocytes having growth factor secretory ability by introducing said modified adnovirus vector into said non-hematocytes.

8. Non-hematocytes having growth factor secretory ability, wherein a gene encoding growth factor is introduced into said non-hematocytes using adenovirus vector.
